(19) United States
(12) Patent Application Publication
(10) Pub. No.: US 2025/0246208 A1
(43) Pub. Date: Jul. 31, 2025

(54) VIDEO AND AUDIO CAPTURE OF CATHLAB PROCEDURES

(71) Applicants: Medtronic Vascular, Inc., Santa Rosa, CA (US); Digital Surgery Ltd., London (GB)

(72) Inventors: Kaitlin Templeton, San Francisco, CA (US); Heidrun Behrmann, Frankfurt (DE); Giordana M. Belenchia, Las Vegas, NV (US); Abiral Joshee, Seattle, WA (US); Benjamin J. Lebow, Healdsburg, CA (US); Federico Saenz Salas, Scottsdale, AZ (US); Kevin Vu, Brooklyn Park, MN (US); Angela Yu, London (GB); Jeffrey Michael Zalewski, Corte Madera, CA (US)

(21) Appl. No.: 18/854,220

(22) PCT Filed: Apr. 7, 2023
(86) PCT No.: PCT/US2023/017887

§ 371 Date: Oct. 4, 2024

Related U.S. Application Data
(60) Provisional application No. 63/362,631, filed on Apr. 7, 2022.

(51) Int. Cl. G11B 27/34; G06F 3/0482; G06V 20/40; G10L 15/08; G11B 27/031; H04N 5/91

(52) U.S. Cl. CPC G11B 27/34; G06F 3/0482; G06V 20/44; G10L 15/08; G11B 27/031; H04N 5/91; G06V 2201/03; G10L 2015/088

(57) ABSTRACT

Example medical systems and techniques are disclosed. In an example, a medical system includes memory configured to store video data and audio data, the video data and audio data being captured during a medical procedure of a patient. The medical system includes processing circuitry communicatively coupled to the memory, the processing circuitry being configured to receive the video data from one or more first sensors and receive the audio data from one or more second sensors. The processing circuitry is also configured to register the video data with the audio data.

This application claims the benefit of U.S. Provisional Patent Application 63/362,631, filed Apr. 7, 2022, and entitled “VIDEO AND AUDIO CAPTURE OF CATHLAB PROCEDURES.”

### TECHNICAL FIELD

This disclosure relates to the capture of video and audio during a medical procedure.

### BACKGROUND

During a medical procedure, a clinician may use an imaging system to be able to visualize internal anatomy of a patient. Such an imaging system may display anatomy, medical instruments, or the like, and may be used to diagnose a patient condition or assist in guiding a clinician in moving a medical instrument to an intended location inside the patient. Imaging systems may use sensors to capture video images which may be displayed during the medical procedure. Imaging systems include ultrasound imaging systems, computed tomography (CT) scan systems, magnetic resonance imaging (MRI) systems, isocentric C-arm fluoroscopic systems, positron emission tomography (PET) systems, as well as other imaging systems.

### SUMMARY

In general, this disclosure is directed to various techniques and medical systems for capturing video and audio data during a medical procedure. Such captured video and audio data may be used to assist a clinician in categorizing the medical procedure or a patient condition, for filling out “paperwork” after or during the medical procedure, for training purposes, to assist with an intraprocedural event, or for performance monitoring.

In one example, the disclosure describes a medical system comprising memory configured to store video data and audio data, the video data and audio data being captured during a medical procedure of a patient; and processing circuitry communicatively coupled to the memory, the processing circuitry being configured to: receive the video data from one or more first sensors; receive the audio data from one or more second sensors; and register the video data with the audio data.

In another example, the disclosure describes a method comprising receiving, by processing circuitry, video data from one or more first sensors, the video data being captured during a medical procedure of a patient; receiving, by processing circuitry, audio data from one or more second sensors, the audio data being captured during the medical procedure of the patient; and registering, by the processing circuitry, the video data with the audio data.

In yet another example, the disclosure describes a non-transitory computer readable medium comprising instructions, which, when executed, cause processing circuitry to receive video data from one or more first sensors, the video data being captured during a medical procedure of a patient; receive audio data from one or more second sensors, the audio data being captured during the medical procedure of the patient; and register the video data with the audio data.

These and other aspects of the present disclosure will be apparent from the detailed description below. In no event, however, should the above summaries be construed as limitations on the claimed subject matter, which subject matter is defined solely by the attached claims.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a schematic perspective view of one example of a system for guiding a medical instrument through a region of a patient.

FIG. 2 is a schematic view of one example of a computing system of the system of FIG. 1.

FIG. 3 is a conceptual diagram of an example home screen of user interface 218 of FIG. 2 according to the techniques of this disclosure.

FIG. 4 is a conceptual diagram illustrating another example page of user interface 218 of FIG. 2.

FIG. 5 is a flow diagram illustrating video and audio collection techniques according to one or more aspects of this disclosure.

FIG. 6 is a conceptual diagram illustrating an example machine learning model according to one or more aspects of this disclosure.

FIG. 7 is a conceptual diagram illustrating an example training process for a machine learning model according to one or more aspects of this disclosure.

### DETAILED DESCRIPTION

Imaging systems may be used to assist a clinician in a medical procedure, such as a diagnostic procedure, an intervention procedure, such as a percutaneous coronary intervention (PCI) procedure, or both. For example, imaging systems may be used to determine lesions within a vasculature of a patient that may be limiting or obstructing blood flow within the patient. Imaging systems may also be used when performing an angioplasty procedure, or other medical procedure intended to treat lesions within the vasculature of the patient. While described primarily herein with respect to the vasculature of a patient, imaging systems described herein may be used for other medical purposes and are not limited to coronary purposes. Imaging systems may generate video data via sensors. This video data may be recorded for later use. The video data may include representations of portions of vasculature of a patient, including one or more lesions which may be restricting blood flow through the portion of the vasculature, a geometry and location within a blood vessel of such lesions, and/or any medical instrument which may be within a field of view of one or more sensors of the imaging system. Aspects of this disclosure are applicable to at least Catheterization lab (Cath lab) procedures. Example Cath lab procedures include, but are not necessarily limited to, coronary procedures, renal denervation (RDN) procedures, structural heart and aortic (SH&A) procedures (e.g., transcatheter aortic valve replacement (TAVR), transcatheter mitral valve replacement (TMVR), and the like), device implantation procedures (e.g., heart monitors, pacemakers, defibrillators, and the like), etc.

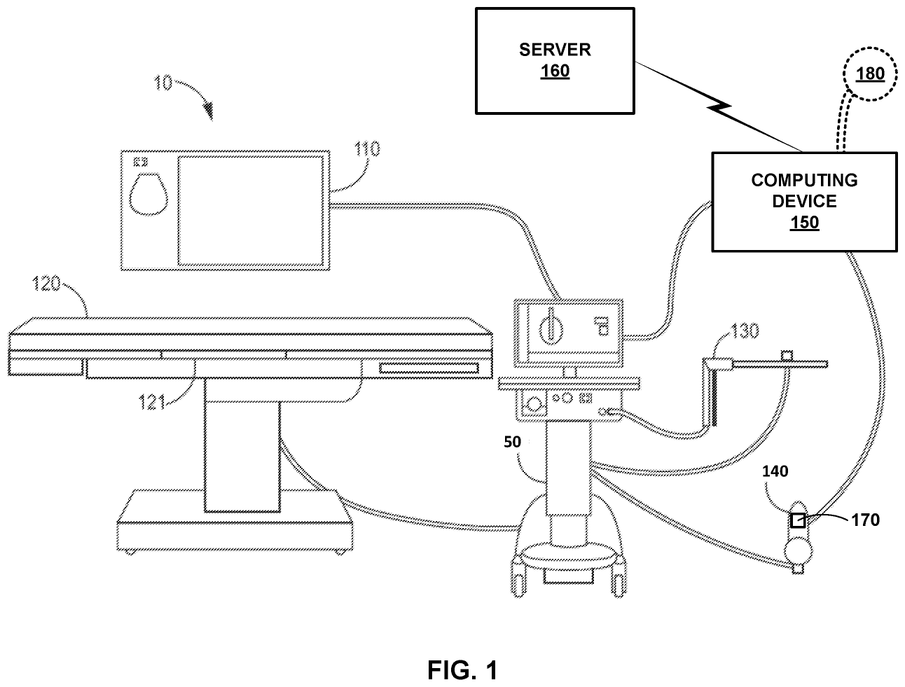

FIG. 1 is a schematic perspective view of one example of a system 10, which includes a guidance workstation 50, a display device 110, a table 120, a medical instrument 130, an imager 140, and a computing device 150. Guidance workstation 50 may include, for example, an off-the-shelf device, such as a laptop computer, desktop computer, tablet computer, smart phone, or other similar device. In some examples, guidance workstation may be a specific purpose device. Guidance workstation 50 may be configured to control an electrosurgical generator, a peristaltic pump, a power supply, or any other accessories and peripheral devices relating to, or forming part of, system 10. Computing device 150 may include, for example, an off-the-shelf device such as a laptop computer, desktop computer, tablet computer, smart phone, or other similar device or may include a specific purpose device.

Display device 110 may be configured to output instructions, images, and messages relating to at least one of a performance, position, orientation, or trajectory of medical instrument 130. Further, the display device 110 may be configured to output information regarding medical instrument 130, e.g., model number, type, size, etc. Table 120 may be, for example, an operating table or other table suitable for use during a medical procedure that may optionally include an electromagnetic (EM) field generator 121. EM field generator 121 may be optionally included and used to generate an EM field during the procedure and, when included, may form part of an EM tracking system that is used to track the positions of one or more medical instruments within the body of a patient. EM field generator 121 may include various components, such as a specially designed pad to be placed under, or integrated into, an operating table or patient bed.

Medical instrument may also be visualized by using imaging, such as ultrasound imaging. In the example of FIG. 1, an imager 140, such as an ultrasound wand, may be used to image the patient's body during the procedure to visualize the locations of medical instruments, such as surgical instruments, device delivery or placement devices, and implants, inside the patient's body. Imager 140 may include one or more sensors 170. For example, imager 140 may include an ultrasound probe having an ultrasound transducer array. In some examples, imager 140 may include an ultrasound transducer array, including a plurality of transducer elements. These transducer elements may be configured to sense ultrasound energy reflected off of anatomy of the patient and/or medical instrument 130. In some examples, one or more sensors 170 may also include one or more sensors configured to capture audio data, such as one or more microphones. In some examples, rather than one or more sensors 170 including one or more sensors configured to capture audio data, computing device 150, or workstation 50 may include one or more sensors configured to capture audio data. Imager 140 may optionally have an EM tracking sensor embedded within or attached to an ultrasound wand or probe, for example, as a clip-on sensor, or a sticker sensor. While described primarily as an ultrasound imager, imager 140 may be any type of imaging device including one or more sensors.

Imager 140 may image a region of interest in the patient's body. The particular region of interest may be dependent on anatomy, the diagnostic procedure, and/or the intended therapy. For example, when performing a PCI, a portion of the vasculature may be the region of interest.

As described further herein, imager 140 may be positioned in relation to medical instrument 130 such that the medical instrument is at an angle to the ultrasound image plane, thereby enabling the clinician to visualize the spatial relationship of medical instrument 130 with the ultrasound image plane and with objects being imaged. Further, if provided, the EM tracking system may also track the location of imager 140. In one or more examples, imager 140 may be placed inside the body of the patient. The EM tracking system may then track the locations of such imager 140 and the medical instrument 130 inside the body of the patient. In some examples, the functions of computing device 150 may be performed by guidance workstation 50 and computing device 150 may not be present.

The location of the medical instrument within the body of the patient may be tracked during the surgical procedure. An exemplary method of tracking the location of the medical instrument includes using the EM tracking system, which tracks the location of medical instrument 130 by tracking sensors attached to or incorporated in medical instrument 130. Prior to starting the procedure, the clinician may verify the accuracy of the tracking system using any suitable technique or techniques. Any suitable medical instrument 130 may be utilized with the system 10. Examples of medical instruments or devices include stents, catheters, angioplasty devices, ablation devices, etc.

Computing device 150 may be communicatively coupled to imager 140, workstation 50, display device 110 and/or server 160, for example, by wired, optical, or wireless communications. Server 160 may be a hospital server, a cloud-based server or the like. Server 160 may be configured to store patient video data, audio data, electronic healthcare or medical records or the like. In some examples, computing device 150 may be an example of workstation 50.

Computing device 150 may be configured to receive video data from one or more sensors 170. Computing device may also be configured to receive audio data from one or more sensors. Such sensors may be part of one or more sensors 170 or may be located elsewhere within system 10, such as within computing device 150 or workstation 50. Such audio data may include significant events during the medical procedure, such as the inflation of an angioplasty balloon, notes to be used when completing “paperwork” relating to the medical procedure or a patient condition, etc. Computing device 150 may be configured to register the video data with the audio data. For example, computing device 150 may apply one or more video timestamps to the video data and apply one or more audio timestamps to the audio data and use the timestamps to synchronize or register the audio data with the video data. In some examples, computing device 150 may register the audio data with the video data substantially in “real time.” In other examples, computing device 150 may register the audio data with the video data, after applying the timestamps, offline, for example, after the medical procedure.

Computing device 150 may also be configured to present a user interface on a display, such as a display of computing device 150 or display device 110. Such a user interface may include a plurality of drop-down menus. Each of these drop-down menus may have a limited number of selection options. A clinician may use the drop-down menus, during or after the medical procedure, to make selections of the selection options, regarding the medical procedure or any observed patient conditions. Computing device 150 may use the selected options to categorize at least one of the medical procedures (e.g., as a diagnostic procedure as opposed to a PCI procedure) or a patient condition (e.g., multiple lesions).

Computing device 150 may also be configured to receive other data from any devices of system 10. For example, computing device 150 may receive data from one or more sensors 180. The received data may represent hemodynamic data or other numeric data. Computing device 150 may register the other data with the first video data from one or more sensors 170. Computing device 150 may then overlay the first video data and the other data.

Computing device 150 may also be configured to receive video data from more than one type of imaging system. For example, one or more sensors 180 may represent a different type of one or more sensors than one or more sensors 170. For example, one or more sensors 170 may include ultrasound sensors, while one or more sensors 180 may include fluoroscopy sensors. Computing device 150 may receive second video data from one or more sensors 180. Computing device 150 may register the second video data from one or more sensors 180 with the first video data from one or more sensors 170. Computing device 150 may then overlay the first video data and the second video data. While shown as separately communicatively coupled to computing device 150, in some examples, both one or more sensors 170 and one or more sensors 180 may be communicatively coupled to computing device 150 via a single connection or feed.

In some examples, computing device 150 may be configured to execute a machine learning model to determine at least one of a patient condition or a type of the medical procedure. For example, the machine learning model may be trained on data from past medical procedures performed on a plurality of patients having different patient conditions. Based on the at least one of the patient condition or the type of the medical procedure, the computing device may automatically generate a report template. This report template may be a labor-saving device designed to facilitate a clinician in filling out “paperwork” relating to the medical procedure and/or the patient condition. For example, computing device 150 may present a user interface on a display including the report template. The clinician may then use the report template within the user interface to complete the “paperwork.” Once this report template has been filled out by the clinician, computing device 150 may store the completed report locally, e.g., in memory of computing device 150, or may upload the completed report to server 160 which may be configured to store such reports in patient electronic healthcare or medical records. In the case where the machine learning mode may determine the patient condition, computing device 150 may additionally, or alternatively, present a user interface on a display including the determined patient condition. In some examples, computing device 150 may use the determined patient condition to generate a recommendation for a clinical trial for the patient. Computing device 150 may present the recommendation to the clinician via a user interface.

Computing device 150 may be configured to share video data captured during a medical procedure with clinicians, including clinicians not present during the medical procedure. For example, a clinician may, via a user interface of computing device 150, tag a video (or a video excerpt) for sharing with one or more other clinicians, predefined groups of people, hospital management, or the like. In some examples, a limited amount of patient data may be associated with the video that may also be shared. Such videos may be used to train clinicians, for performance review purposes, or the like. For example, computing device 150 may receive, via a user interface, performance information relating to the medical procedure and associate that performance information with one or more clinicians that performed the medical procedure. Computing device 150 may store the performance information in memory of computing device 150 or upload the performance information to memory of server 160.

Computing device 150 may be configured to upload at least one of the video data or the audio data to server 160. Computing device 150 may also be configured to edit the video data to generate an excerpt of the video data. Computing device 150 may store the excerpt of the video data in memory, e.g., local memory of computing device 150. In some examples, after generating the excerpt of the video data, computing device 150 may erase any locally stored copy of the full video data of the procedure. The video excerpts may be automatically generated by computing device 150 based on events occurring during the medical procedure which may be pinned or otherwise identified in a user interface by a clinician or based on events identified from the audio data (such as by using natural language processing) and/or video data (by using a computer vision model and/or a machine learning model). Alternatively, or additionally, a clinician may generate a video excerpt by editing the video data themselves, via a user interface.

Computing device 150 may be configured to execute a computer vision model to determine an intraprocedural event. For example, the computer vision model may be trained to recognize an end-of-life medical instrument (e.g., medical instrument 130) or an error by a clinician during a medical procedure based on the video data being received by computing device 150. In some examples, computing device 150 may be configured to take an action based on determining the intraprocedural event. For example, computing device 150 may inform, via a user interface, a clinician to save the medical instrument and/or generate a shipping label which may be used to return the medical instrument.

In some examples, computing device 150 may not record audio data throughout the entire medical procedure. For example, computing device 150 may be configured to check for a wake-up word in received audio data from, for example one or more microphones. For example, a clinician may state “wake-up” and computing device 150 may be configured to, based on receiving the wake-up word “wake-up” from one or more sensors (e.g., microphones) begin recording the audio data to memory. Computing device 150 may also be configured to only record the audio data for a predetermined time period, for example, some amount of time in the range of ten seconds to five minutes, such as thirty seconds. As such, computing device 150 may determine that the predetermined time period from receiving the wake-up word has expired, and based on the determination that the predetermine time period from receiving the wake-up word has expired, stop recording the audio data to the memory. In some examples, computing device 150 may generate an indication that the recording is going to stop before stopping the recording. For example, computing device 150 may output via a user interface, such an indication. The indication may be a visual indication displayed on a display, such as “audio recording is ending” or an audible indication played over a speaker such as a beep or a phrase such as “audio recording is ending.”

In some examples, computing device 150 may continue to record the audio data until a clinician states a “go-to-sleep” word. For example, a clinician may state “Go-to-sleep” and computing device 150 may be configured to, based on receiving the go-to-sleep word “go-to-sleep” from one or more sensors (e.g., microphones) stop recording the audio data to memory. It should be noted that a wake-up word and/or a go-to-sleep word may include more than a single word.

If a clinician would like to record audio later in the medical procedure, the clinician may again invoke the wake-up word to begin recording again. In this manner, computing device 150 may record more important audio information relating to the medical procedure or a patient condition, while maintaining some amount of audio privacy for the clinician(s) and/or patient. In some examples, the memory is local to computing device 150. In some examples, the memory may be within any of one or more components of system 10. The one or more microphones may be local to computing device 150 or may be part of any of one or more components of system 10.

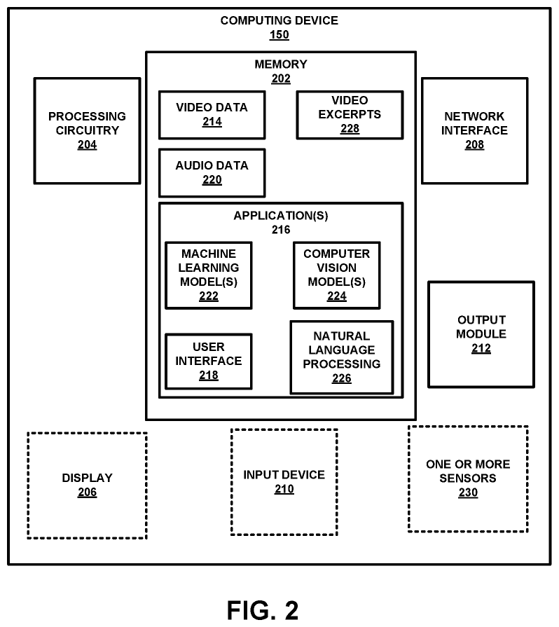

FIG. 2 is a schematic view of one example of a computing device 150 of system 10 of FIG. 1. Computing device 150 may include a workstation, a desktop computer, a laptop computer, a smart phone, a tablet, a dedicated computing device, or any other computing device capable of performing the techniques of this disclosure.

Computing device 150 may be configured to perform processing, control and other functions associated with guidance workstation 50, imager 140, and an optional EM tracking system. As shown in FIG. 2, computing device 150 represents multiple instances of computing devices, each of which may be associated with one or more of guidance workstation 50, imager 140, or the EM tracking system. Computing device 150 may include, for example, a memory 202, processing circuitry 204, a display 206, a network interface 208, an input device 210, or an output module 212, each of which may represent any of multiple instances of such a device within the computing system, for ease of description.

While processing circuitry 204 appears in computing device 150 in FIG. 2, in some examples, features attributed to processing circuitry 204 may be performed by processing circuitry of any of computing device 150, guidance workstation 50, imager 140, or the EM tracking system, or combinations thereof. In some examples, one or more processors associated with processing circuitry 204 in computing system may be distributed and shared across any combination of computing device 150, guidance workstation 50, imager 140, and the EM tracking system. Additionally, in some examples, processing operations or other operations performed by processing circuitry 204 may be performed by one or more processors residing remotely, such as one or more cloud servers or processors, each of which may be considered a part of computing device 150. Computing device 150 may be used to perform any of the methods described in this disclosure, and may form all or part of devices or systems configured to perform such methods, alone or in conjunction with other components, such as components of computing device 150, guidance workstation 50, imager 140, an EM tracking system, or a system including any or all of such systems.

Memory 202 of computing device 150 includes any non-transitory computer-readable storage media for storing data or software that is executable by processing circuitry 204 and that controls the operation of computing device 150, guidance workstation 50, imager 140, or EM tracking system, as applicable. In one or more examples, memory 202 may include one or more solid-state storage devices such as flash memory chips. In one or more examples, memory 202 may include one or more mass storage devices connected to the processing circuitry 204 through a mass storage controller (not shown) and a communications bus (not shown).

Although the description of computer-readable media herein refers to a solid-state storage, it should be appreciated by those skilled in the art that computer-readable storage media may be any available media that may be accessed by the processing circuitry 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium that may be used to store the desired information and that may be accessed by computing device 150. In one or more examples, computer-readable storage media may be stored in the cloud or remote storage and accessed using any suitable technique or techniques through at least one of a wired or wireless connection.

Memory 202 may store video data 214 and audio data 220. Video data 214 may be captured by one or more sensors 170 (FIG. 1) during a medical procedure of a patient. Processing circuitry 204 may receive video data 214 from one or more sensors 170 and store video data 214 in memory 202. Audio data 220 may be captured by one or more sensors 170 or by one or more sensors 230. One or more sensors 170 or one or more sensors 230 may include one or more microphones. Processing circuitry 204 may receive audio data 220 from one or more sensors 170 or one or more sensors 230 and may store audio data 220 in memory 202. Processing circuitry 204 may execute user interface 218 so as to cause display 206 (and/or display device 110 of FIG. 1) to present user interface 218 to one or more clinicians performing the medical procedure. Memory 202 may also store one or more machine learning models 222, one or more computer vision modules 224, natural language processing engine 226, and user interface 218. One or more machine learning models 222 may be configured to, when executed by processing circuitry 204, determine at least one of a patient condition or a type of the medical procedure. Processing circuitry 204 may use the at least one of the patient condition or the type of the medical procedure to, for example, generate a report template for a clinician to streamline or partially fill out “paperwork” associated with the medical procedure, to generate a recommendation for a clinical trial for the patient, to identify a patient condition, to determine how to edit a video to generate a video excerpt, or the like. In some implementations, display 206 may be located external to computing device 150. One or more computer vision models 224 may be configured to, when executed by processing circuitry 204, determine an intraprocedural event, such as the breaking of a medical instrument or an error of the clinician or to determine how to edit a video to generate a video excerpt. In the case of determining an intraprocedural event, processing circuitry 204 may use such determination of the intraprocedural event to take an action, such as inform a clinician, e.g., via user interface 218, to save the medical instrument, and/or to generate a shipping label. Processing circuitry 204 may execute natural language processing engine 226 when listening for a wake-up word or to identify significant events during the medical procedure based on audio data 220 which processing circuitry 204 may use to edit video data 214 to generate video excerpts 228.

During the medical procedure the clinician may begin recording video and/or audio. User interface 218 may include tags, such as “complication,” “stent placement,” “additional device,” “other” for example. In some examples, when selecting the tag, processing circuitry 204 may apply a timestamp associating the selection with video data 214 and/or audio data 220.

In some examples, once the medical procedure is completed, user interface 218 may ask a clinician for a title, type of procedure, and patient condition information, such as number of lesions, lesion attributes, and lesion location. In some examples, one or more of these (e.g., type of procedure and patient condition information) are posited to the clinician via drop-down menus which each may have a limited number of possible selections. The Such selections (which may also be referred to herein as tags or pins) may serve to classify the medical procedure and/or a patient condition and may be used to find the associated video data and/or audio data or other information relating to the procedure via a search at a later time. By using a limited number of tags, the tags may be used to train one or more machine learning model(s) 222 and/or one or more computer vision model(s) 224 together with video data 214 and/or audio data 220 such that one or more machine learning model(s) 222 and/or one or more computer vision model(s) 224 may learn to apply the tags based on video data 214 and/or audio data 220. In this manner, after sufficient training, processing circuitry 204 may apply the tags automatically by executing one or more machine learning model(s) 222 and/or one or more computer vision model(s) 224.

In some examples, user interface 218 may permit a clinician to view videos or video excerpts of their medical procedures and videos or video excerpts of other medical procedures which have been shared with them. In some examples, user interface 218 include messaging functionality allowing a clinician to share insights with other clinicians. In some examples, the videos presented by user interface 218 may be video excerpts 228. User interface may provide links to the complete videos (e.g., video data 214) which may be stored on server 160.

In some examples, user interface 218 may use two factor authentication to permit a clinician to use email or text functionality. In some examples, the user interface may present CAPTCHA like tests on still images, such as images from fluoroscopy, with which a clinician may complete. These completed tests may be used to train one or more machine learning models 222 and/or one or more computer vision models 224.

Processing circuitry 204 may be implemented by one or more processors, which may include any number of fixed-function circuits, programmable circuits, or a combination thereof. As described here, guidance workstation 50 may perform various control functions with respect to imager 140 and may interact extensively computing device 150. Guidance workstation 50 may be communicatively coupled to computing device 150, enabling guidance workstation 50 to control the operation of imager 140 and receive the output of imager 140. In some examples, computing device 150 may control various operations of imager 140.

In various examples, control of any function by processing circuitry 204 may be implemented directly or in conjunction with any suitable electronic circuitry appropriate for the specified function. Fixed-function circuits refer to circuits that provide particular functionality and are preset on the operations that may be performed. Programmable circuits refer to circuits that may programmed to perform various tasks and provide flexible functionality in the operations that may be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Instructions may be executed by one or more processors, such as one or more digital signal processors (DSPs), general purpose microprocessors, application specific integrated circuits (ASICs), field programmable gate arrays (FPGAs), graphics processing units (GPUs) or other equivalent integrated or discrete logic circuitry. Accordingly, the term processing circuitry 204 as used herein may refer to one or more processors having any of the foregoing processor or processing structure or any other structure suitable for implementation of the techniques described herein. In addition, in some aspects, the functionality described herein may be provided within dedicated hardware or software modules configured for encoding and decoding, or incorporated in a combined codec. Also, the techniques could be fully implemented in one or more circuits or logic elements.

Display 206 may be touch sensitive or voice activated (e.g., via one or more sensors 230 which may include one or more microphones), enabling display 206 to serve as both an input and output device. Alternatively, a keyboard (not shown), mouse (not shown), or other data input devices (e.g., input device 210) may be employed.

Network interface 208 may be adapted to connect to a network such as a local area network (LAN) that includes a wired network or a wireless network, a wide area network (WAN), a wireless mobile network, a Bluetooth network, or the internet. For example, guidance workstation 50 and/or computing device 150 may receive video data from one or more sensors 170 during a medical procedure. Guidance workstation 50 and/or computing device 150 may receive updates to its software, for example, application 216, via network interface 208. Guidance workstation 50 and/or computing device 150 may also display notifications on display 206 that a software update is available.

Input device 210 may be any device that enables a user to interact with guidance workstation 50 and/or computing device 150, such as, for example, a mouse, keyboard, foot pedal, touch screen, augmented-reality input device receiving inputs such as hand gestures or body movements, or voice interface.

Output module 212 may include any connectivity port or bus, such as, for example, parallel ports, serial ports, universal serial busses (USB), or any other similar connectivity port known to those skilled in the art.

Application(s) 216 may be one or more software programs stored in memory 202 and executed by processing circuitry 204 of computing device 150. Processing circuitry 204 may execute user interface 218, which may display video data 214 on display 206 and/or display device 110. Video data 214 may be stored for future use, such as training and/or performance review of clinicians performing the medical procedure. In some examples, processing circuitry 204 may, based on events occurring during the medical procedure or based on clinician input into user interface 218, edit video data 214 to generate video excerpts 228. In some examples, processing circuitry 204 may communicate with server 160 (FIG. 1) to upload video data 214 during or after the medical procedure. In some examples, after generating video excerpts 228 and uploading video data 214 to server 160, processing circuitry 204 may remove video data 214 from computing device 150.

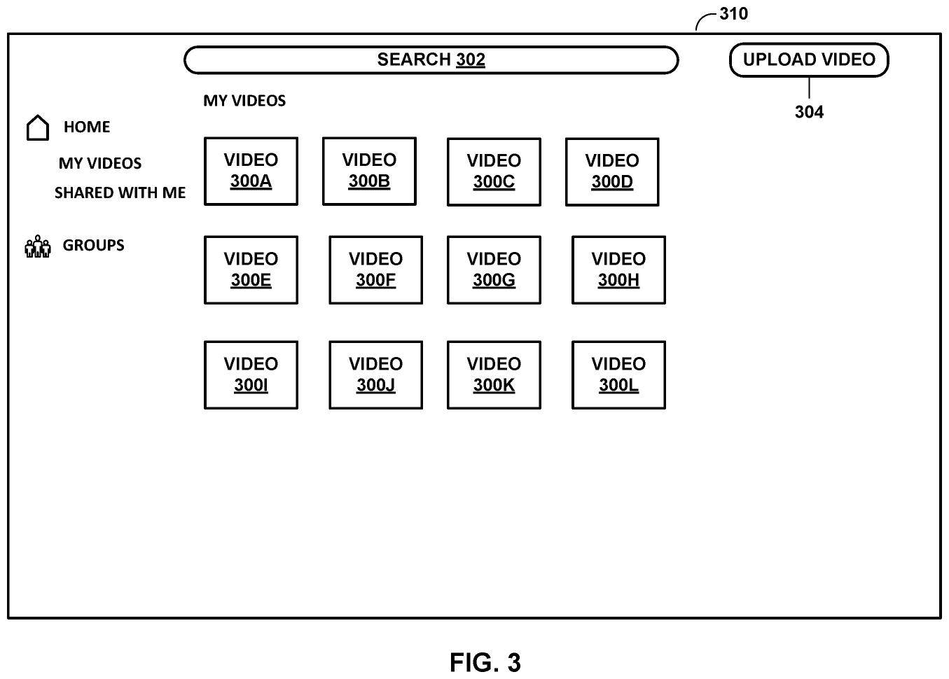

FIG. 3 is a conceptual diagram of an example home screen of user interface 218 of FIG. 2 according to the techniques of this disclosure. For example, home screen 310 may be a home screen for a particular clinician. To reach home screen 310, the clinician may need to select their name from a drop-down list of clinicians and enter a password. Home screen 310 may include a plurality of videos 300A-300L (collectively “videos 300”), search bar 302, and upload video button 304. The term “button” as used herein includes virtual buttons, icons or the like. Videos 300 may be videos or video excerpts of medical procedures performed by a given clinician. By selecting a video of videos 300, the clinician may view the particular video or may then click on upload video button 304 to upload the particular video, for example, to server 160. The terms “click” and “clicking” as used herein are not meant to be restrictive, but are meant to convey that the clinician may make a selection through a user interface via the use of a touchscreen, a mouse, a stylus, or the like. The clinician may also search through videos available to them by entering search terms, such as tags or the like, into search bar 302. If the clinician were to click on “shared with me,” videos 300 would not be displayed and in their places would be any videos that have been shared with the clinician. While not shown in FIG. 3 for simplicity purposes, in the area above or below each of videos 300 there may be displayed information relating to the video, such as a title of the procedure, a date of the procedure, the type of procedure (e.g., diagnostic only, diagnostic and PCI, or PCI only, or the like), the name of the clinician who performed the procedure, and/or patient condition information (e.g., single lesion, multiple lesions, lesion attributes (e.g., diffuse disease, bifurcates, chronic total occlusion, modest calcification, etc.), lesion location, or the like). Such information may assist the clinician in more easily finding a particular video.

In some examples, user interface 218 may employ groups, which a clinician may access from home page 310 by clicking on the groups icon. Such groups may be based on which facility clinicians are using, Fellows, Attending clinicians (e.g., Attending physicians), which type of procedure the clinician typically performs, or any other type of grouping.

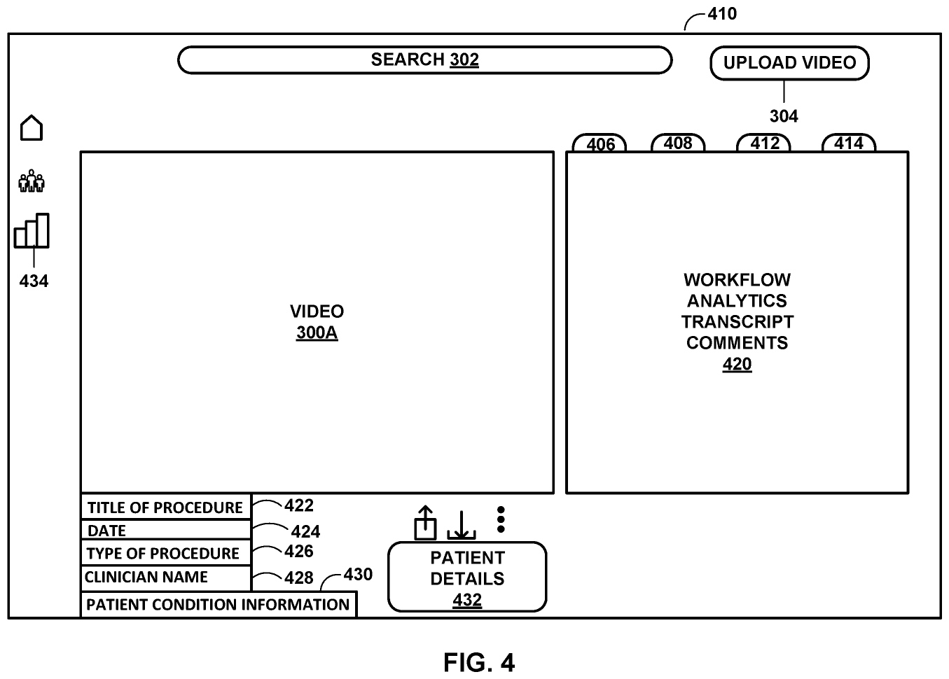

FIG. 4 is a conceptual diagram illustrating another example page of user interface 218 of FIG. 2. Page 410 may represent a user interface page that replaces home page 310 when a clinician clicks on a particular video of videos 300. Page 410 may retain search bar 302 and upload video button 304. Page 410 may display an enlarged and/or higher resolution video of the selected video from home page 310, for example, video 300A. The clinician may click in video 300A and enter objectives (discussed later) to skip to a portion of interest of video 300A. In some examples, video 300A may include a slider, play button, forward button, reverse button, pause button, or the like for controlling the playback of video 300A.

During or after a medical procedure, processing circuitry 204 via page 410 may prompt the clinician to enter a title of the procedure in a title of procedure field 422. For example, the clinician may enter the title via a keyboard of input device 210 (FIG. 2) or a virtual keyboard of page 410 (not shown) which may pop up when title of the procedure field 422 is clicked on or touched. During or after the medical procedure, processing circuitry 204 via page 410 may prompt the clinician to enter a date in date field 424 or may automatically fill in the date in date field 424. During or after the medical procedure, processing circuitry 204 via page 410 may prompt the clinician to enter a type of procedure. This type of procedure may be entered via drop-down menu 426 having a limited number of choices or tags. During or after the procedure, processing circuitry 204 via page 410 may prompt the clinician to enter patient condition information. Patient condition information may be entered via one or more drop-down menus 430 each having a limited number of choices or tags. For example, there may be a single drop-down menu for patient condition information or separate drop-down menus for different aspects of the patient condition, such as number of lesions, lesion location, lesion attributes, etc. Page 410 may also display the clinician's name. The clinician may not be required to enter their name as they may already be logged in and processing circuitry 204 may automatically populate the clinician name field.

Page 410 may include workflow, analytics, transcript, and/or comments which may be displayed in workflow analytics transcript comments area 420. Each of these may be separately accessed by a clinician by clicking on a tab or button, such tabs 406, 408, 412, and 414 respectively. For example, clicking on the workflow tab may bring up workflow information in workflow analytics transcript comments area 420. Workflow information may include objectives, key steps, and/or notes. Processing circuitry 204 may associate a time stamp for each objective or key step entered when they are achieved, and page 410 may display such time stamp along with the objective or key step. Example objectives or key steps may include, target vessel access, lesion assessment, stent deployment, etc.

The clinician may add notes regarding the procedure via page 410 in workflow analytics transcript comments area 420. For example, the clinician may add a note regarding the lesion assessment objective that the lesion is hemodynamically significant.

In some examples, page 410 may display different data types in a combined manner. For example, processing circuitry 204 may control user interface 218 to display intravascular ultrasound, optical coherence tomography, fractional flow reserve, and/or other date types simultaneously in video 300A. For example, processing circuitry 204 may apply timestamps to each of these types of data and overlay these different types of data for display on video 300A of page 410.

Processing circuitry 204 may measure time taken to perform each step during the procedure which processing circuitry 204 may display via user interface 218. In some examples, processing circuitry 204 may determine an average of time taken to perform each step and may display a comparison of time taken to perform each step of the current medical procedure to the average time taken to perform each step of previous medical procedures. In some examples, this average is specific to the clinician performing the current medical procedure. In some examples, this average is based on one or more groups of clinicians of which the clinician performing the current medical procedure is a member. In some examples, a clinician may access such information in the form of a bar graph or other graph by clicking on graph icon 434 or by clicking on the analytics tab. For example, such graphs may be displayed in a pop-up menu or in workflow analytics transcript comments area 420.

In some examples, user interface 218 may display a transcript of recorded audio in response to the clinician clicking on the transcript tab. For example, processing circuitry 204 may execute natural language processing engine 226 to determine what is being said, translate the speech into text, and display a transcript of the text in workflow analytics transcript comments area 420. An example comment may be from a Fellow clinician to an Attending clinician “grant view-only access to Attending, send notification to Attending to allow coaching.”

User interface 218 may include an option to export or upload video data 214, audio data 220, video excerpts 228, and/or other collected information (e.g., tags, transcript, notes, etc.) to an electronic medical record which may be stored in server 160. For example, a clinician may select upload video button 304 to export or upload such information to server 160. Additionally, user interface 218 may provide an option to share such information with other clinicians, groups or people, hospital management, or the like.

Page 410 may include patient details button by which a clinician may view patient details, for example, via a pop-up window. Such patient details may be limited, for example, an identifier number, age, sex, body mass index, and/or clinical history. Such information may be useful in searching for similar cases. In some examples, page 410 or the pop-up window may also include a link to that patient's electronic medical records which may be stored in server 160.

The techniques of this disclosure may be used for training purposes, to provide coaching at an administrative level to clinicians, and for facilitating the timely transfer of information between clinicians. For example, if a clinician is a performing a diagnostic medical procedure or if a clinician is performing a medical procedure that is too complicated for their skill level, the patient may be referred to a clinician capable of performing a medical intervention. The clinician who is to perform the medical intervention may receive the patient's file, including the video taken from the first procedure (e.g., the diagnostic procedure). The clinician would be able to view the diagnostic video and search for similar cases. The clinician may view the similar videos to help plan the procedure. The clinician may also tag the videos while watching them and use the tags to search for additional videos.

In some examples, processing circuitry 204 executing one or more machine learning model(s) 222 and/or one or more computer vision model(s) 224 may identify characteristics of lesions, pull data from the patient's electronic medical records, and/or identify similar videos to assist the clinician in planning the medical intervention.

In some examples, processing circuitry 204 may provide real-time clinical guidance to a clinician. For example, processing circuitry 204 may use one or more computer visions model(s) 224 to measure a size of a lesion and/or determine a location of a lesion and provide the clinician with procedure recommendations.

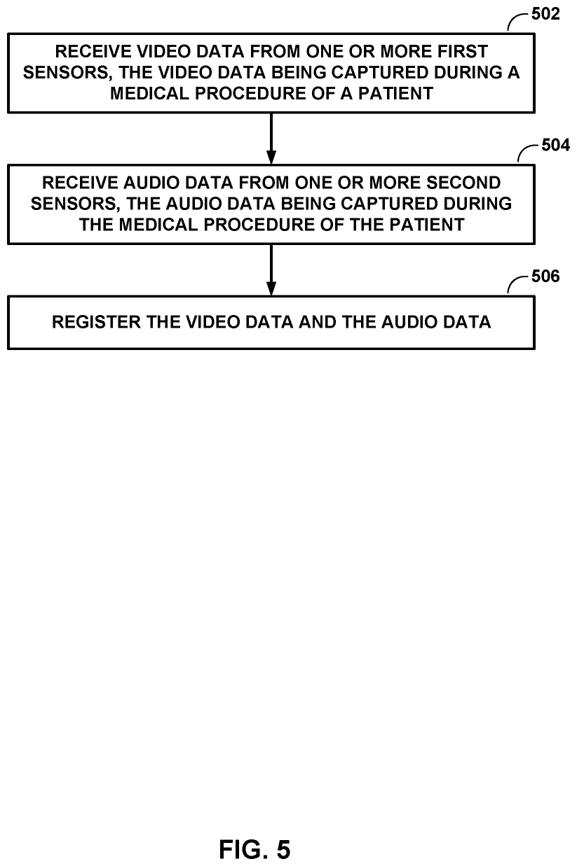

FIG. 5 is a flow diagram of example video and audio capture techniques according to one or more aspects of this disclosure. Processing circuitry 204 may receive video data from one or more first sensors, the video data being captured during a medical procedure of a patient (502). For example, the patient may be undergoing a medical procedure which may be diagnostic, an intervention procedure (such as a PCI), or both. Processing circuitry 204 may receive the video data from one or more image sensors of an imaging system (e.g., an ultrasound imaging system, an isocentric C-arm fluoroscopy system, a PET system, a CT scan system, am MRI system, or the like).

Processing circuitry 204 may receive audio data from one or more second sensors, the audio data being captured during the medical procedure of the patient (504). For example, processing circuitry 204 may receive the audio data from one or more microphones. The one or more microphones may be part of the imaging system, be separate from the imaging system (e.g., on a cellular telephone), or both. This audio data may be captured during the same medical procedure as the video data.

Processing circuitry 204 may register the video data with the audio data (506). For example, processing circuitry 204 may apply one or more video timestamps to the video data and one or more audio timestamps to the audio data and processing circuitry 204 may use the timestamps to synchronize or register the video data with the audio data.

In some examples, processing circuitry 204 may present user interface 218 on display 206 and/or display device 110, wherein the user interface comprises a plurality of drop-down menus, each of the plurality of drop-down menus having a limited number of selection options, the selection options being configured to categorize at least one of the medical procedure or a patient condition.

In some examples, processing circuitry 204 is further configured to receive other data, the other data including at least one of hemodynamic data or other numeric data. Processing circuitry 204 may register the other data with the first video data and overlay the first video data and the other data.

In some examples, the video data is first video data and processing circuitry 204 may receive second video data from one or more third sensors. Processing circuitry 204 may register the second video data with the first video data and overlay the first video data and the second video data on a display. In some examples, the one or more third sensors are of a different type than the one or more first sensors. For example, if the one or more first sensors are ultrasound sensors, the one or more third sensors may be fluoroscopy sensors.

In some examples, processing circuitry 204 may execute a machine learning model to determine at least one of a patient condition or a type of the medical procedure. Based on the at least one of the patient condition or the type of the medical procedure, processing circuitry 204 may automatically generate a report template. Processing circuitry 204 may present a user interface on a display, the user interface including the report template. Such techniques may assist a clinician by reducing the time it may take the clinician to fill out “paperwork” after or during a medical procedure.

In some examples, processing circuitry 204 may execute a computer vision model to determine an intraprocedural event. Based on determining the intraprocedural event, processing circuitry 204 may take at least one action. In some examples, the at least one action includes at least one of informing, via user interface 218 a clinician to save a medical instrument or generating a shipping label.

In some examples, processing circuitry 204 may receive a wake-up word via the second sensor. Based on receiving the wake-up word, processing circuitry 204 may begin to record the audio data to the memory.

In some examples, processing circuitry 204 may determine that a predetermined time period from receiving the wake-up word has expired. Based on the determination that the predetermine time period from receiving the wake-up word has expired, processing circuitry 204 may stop the recording the audio data to memory 202.

In some examples, processing circuitry 204 may receive a go-to-sleep word via the second sensor. Based on receiving the go-to-sleep word, processing circuitry 204 may stop recording the audio data to the memory.

In some examples, processing circuitry 204 may execute a machine learning model to determine a patient condition. Processing circuitry 204 may presenting user interface 218 on a display (e.g., display 206 or display device 110), user interface 218 including the determined patient condition.

In some examples, processing circuitry 204 may share at least one of the video data or the audio data with another person or a group of people (e.g., someone other than the clinician(s) performing the medical procedure). In some examples, processing circuitry 204 may receive, via user interface 218, performance information relating to the medical procedure. Processing circuitry 204 may associate the performance information with one or more clinicians that performed the medical procedure. Processing circuitry 204 may store the performance information in memory 202.

In some examples, processing circuitry 204 may upload at least one of the video data or the audio data to server 160. Processing circuitry 204 may edit the video data to generate an excerpt of the video data. Processing circuitry 204 may store the excerpt of the video data in memory 202.

In some examples, processing circuitry 204 may execute a machine learning model to determine a patient condition. Based on the patient condition, processing circuitry 204 may generate a recommendation for a clinical trial for the patient. Processing circuitry 204 may present, via user interface 218, the recommendation to a clinician.

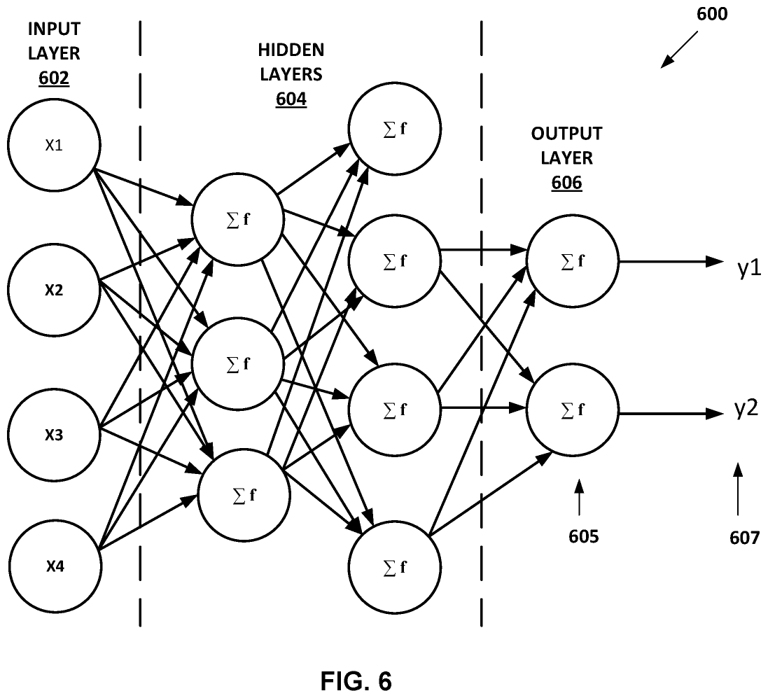

FIG. 6 is a conceptual diagram illustrating an example machine learning model according to one or more aspects of this disclosure. Machine learning model 600 may be an example of the machine learning model(s) 222. In some examples, machine learning model 600 may be a part of computer vision model 224 and/or natural language processing engine 226 discussed above with respect to FIG. 2. Machine learning model 600 may be an example of a deep learning model, or deep learning algorithm, trained to determine a patient condition and/or a type of medical procedure. One or more of computing device 150 and/or server 160 may train, store, and/or utilize machine learning model 600, but other devices of system 10 may apply inputs to machine learning model 600 in some examples. In some examples, other types of machine learning and deep learning models or algorithms may be utilized in other examples. For examples, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

As shown in the example of FIG. 6, machine learning model 600 may include three types of layers. These three types of layers include input layer 602, hidden layers 604, and output layer 606. Output layer 606 comprises the output from the transfer function 605 of output layer 606. Input layer 602 represents each of the input values X1 through X4 provided to machine learning model 600. In some examples, the input values may include any of the of values input into the machine learning model, as described above. For example, the input values may include video data 214 and/or audio data 220, as described above. In addition, in some examples input values of machine learning model 600 may include additional data, such as other data that may be collected by or stored in system 10.

Each of the input values for each node in the input layer 602 is provided to each node of a first layer of hidden layers 604. In the example of FIG. 6, hidden layers 604 include two layers, one layer having four nodes and the other layer having three nodes, but fewer or greater number of nodes may be used in other examples. Each input from input layer 602 is multiplied by a weight and then summed at each node of hidden layers 604. During training of machine learning model 600, the weights for each input are adjusted to establish the relationship between video data 214 and/or audio data 220 and a patient condition and/or a type of medical procedure. In some examples, one hidden layer may be incorporated into machine learning model 600, or three or more hidden layers may be incorporated into machine learning model 600, where each layer includes the same or different number of nodes.

The result of each node within hidden layers 604 is applied to the transfer function of output layer 606. The transfer function may be liner or non-linear, depending on the number of layers within machine learning model 600. Example non-linear transfer functions may be a sigmoid function or a rectifier function. The output 607 of the transfer function may be a classification that video data 214 and/or audio data 220 is indicative of a particular patient condition and/or a particular type of medical procedure.

As shown in the example above, by applying machine learning model 600 to input data such as video data 214 and/or audio data 220, processing circuitry 204 is able to determine a patient condition and/or a type of medical procedure. This may facilitate the planning of medical procedures, the filling out of reports, or the like.

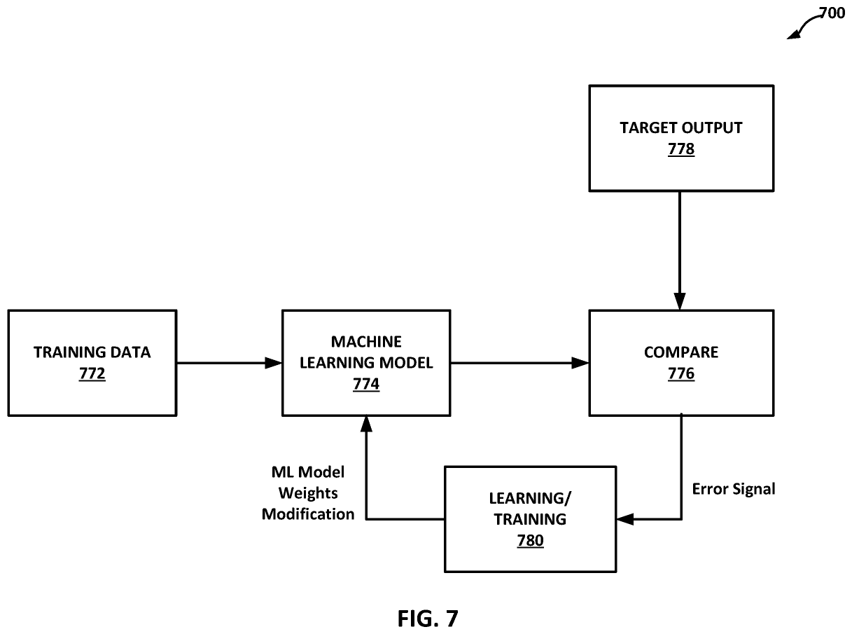

FIG. 7 is a conceptual diagram illustrating an example training process for a machine learning model according to one or more aspects of this disclosure. Process 700 may be used to train machine learning model(s) 222, computer vision model 224, and/or natural language processing engine 226. A machine learning model 774 (which may be an example of machine learning model 600 and/or machine learning model(s) 222) may be implemented using any number of models for supervised and/or reinforcement learning, such as but not limited to, an artificial neural network, a decision tree, naïve Bayes network, support vector machine, or k-nearest neighbor model, CNN, RNN, LSTM, ensemble network, to name only a few examples. In some examples, one or more of computing device 150 and/or server 160 initially trains machine learning model 774 based on a corpus of training data 772. Training data 772 may include, for example, data from past medical procedures performed on a plurality of patients having different patient conditions, tags, video data 214, audio data 220, completed tests, other training data mentioned herein, and/or the like.

While training machine learning model 774, processing circuitry of system 2 may compare 776 a prediction or classification with a target output 778. Processing circuitry 204 may utilize an error signal from the comparison to train (learning/training 780) machine learning model 774. Processing circuitry 204 may generate machine learning model weights or other modifications which processing circuitry 204 may use to modify machine learning model 774. For examples, processing circuitry 204 may modify the weights of machine learning model 600 based on the learning/training 480. For example, one or more of computing device 150 and/or server 160, may, for each training instance in training data 772, modify, based on training data 772, the manner in which a patient condition and/or type of medical procedure is determined.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The terms “controller”, “processor”, or “processing circuitry” may generally refer to any of the foregoing logic circuitry, alone or in combination with other logic circuitry, or any other equivalent circuitry. A control unit comprising hardware may also perform one or more of the techniques of this disclosure. Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), or electronically erasable programmable read only memory (EEPROM), or other computer readable media.

This disclosure includes the following non-limiting examples.

Example 1. A medical system comprising: memory configured to store video data and audio data, the video data and audio data being captured during a medical procedure of a patient; and processing circuitry communicatively coupled to the memory, the processing circuitry being configured to: receive the video data from one or more first sensors; receive the audio data from one or more second sensors; and register the video data with the audio data.

Example 2. The medical system of claim 1, wherein as part of registering the video data with the audio data, the processing circuitry is further configured to: apply one or more video timestamps to the video data; and apply one or more audio timestamps to the audio data.

Example 3A. The medical system of claim 1 or claim 2, wherein the processing circuitry is further configured to present a user interface on a display, wherein the user interface comprises a plurality of drop-down menus, each of the plurality of drop-down menus having a limited number of selection options, the selection options being configured to categorize at least one of the medical procedure or a patient condition.

Example 3B. The medical system of any of claims 1-3A, wherein the processing circuitry is further configured to receive other data, the other data comprising at least one of hemodynamic data or other numeric data; register the other data with the first video data; and overlay the first video data and the other data.

Example 4. The medical system of any of claims 1-3B, wherein the video data is first video data and wherein the processing circuitry is further configured to: receive second video data from one or more third sensors; register the second video data with the first video data; and overlay the first video data and the second video data on a display, wherein one or more third sensors are of a different type than the one or more first sensors.

Example 5. The medical system of any of claims 1-4, wherein the processing circuitry if further configured to: execute a machine learning model to determine at least one of a patient condition or a type of the medical procedure; based on the at least one of the patient condition or the type of the medical procedure, automatically generate a report template; and present a user interface on a display, the user interface comprising the report template.

Example 6. The medical system of any of claims 1-5, wherein the processing circuitry is further configured to: execute a computer vision model to determine an intraprocedural event; and based on determining the intraprocedural event, take at least one action.

Example 7. The medical system of claim 6, wherein the at least one action comprises at least one of: informing, via a user interface, a clinician to save a medical instrument; or generating a shipping label.

Example 8. The medical system of any of claims 1-7, wherein the processing circuitry is further configured to: receive a wake-up word via the second sensor; and based on receiving the wake-up word, begin recording the audio data to the memory.

Example 9A. The medical system of claim 8, wherein the processing circuitry is further configured to: determine that a predetermined time period from receiving the wake-up word has expired; and based on the determination that the predetermine time period from receiving the wake-up word has expired, stop recording the audio data to the memory.

Example 9B. The medical system of claim 8, wherein the processing circuitry is further configured to: receive a go-to-sleep word via the second sensor; and based on receiving the go-to-sleep word, stop recording the audio data to the memory.

Example 10. The medical system of any of claims 1-9, wherein the processing circuitry is further configured to: execute a machine learning model to determine a patient condition; and present a user interface on a display, the user interface comprising the determined patient condition.

Example 11. The medical system of any of claims 1-10, wherein the processing circuitry is further configured to share at least one of the video data or the audio data with another person or a group of people.

Example 12. The medical system of any of claims 1-11, wherein the processing circuitry is further configured to: receive, via a user interface, performance information relating to the medical procedure; associate the performance information with one or more clinicians that performed the medical procedure; and store the performance information in the memory.

Example 13. The medical system of any of claims 1-12, wherein the processing circuitry is further configured to: upload at least one of the video data or the audio data to a server; edit the video data to generate an excerpt of the video data; and store the excerpt of the video data in the memory.

Example 14. The medical system of any of claims 1-13, wherein the processing circuitry if further configured to: execute a machine learning model to determine a patient condition; based on the patient condition, generate a recommendation for a clinical trial for the patient; and present the recommendation, via a user interface, to a clinician.

Example 15. A method comprising: receiving, by processing circuitry, video data from one or more first sensors, the video data being captured during a medical procedure of a patient; receiving, by processing circuitry, audio data from one or more second sensors, the audio data being captured during the medical procedure of the patient; and registering, by the processing circuitry, the video data with the audio data.

Example 16. The method of claim 15, wherein registering the video data with the audio data comprises: applying, by the processing circuitry, one or more video timestamps to the video data; and applying, by the processing circuitry, one or more audio timestamps to the audio data.

Example 17. The method of claim 15 or claim 16, further comprising presenting, by the processing circuitry, a user interface on a display, wherein the user interface comprises a plurality of drop-down menus, each of the plurality of drop-down menus having a limited number of selection options, the selection options being configured to categorize at least one of the medical procedure or a patient condition.

Example 18. The method of any of claims 15-17, wherein the video data is first video data and wherein the method further comprises: receiving, by the processing circuitry, second video data from one or more third sensors; registering, by the processing circuitry, the second video data with the first video data; and overlaying, by the processing circuitry, the first video data and the second video data on a display, wherein the one or more third sensors are of a different type than the one or more first sensors.

Example 19. The method of any of claims 15-18, further comprising: executing, by the processing circuitry, a machine learning model to determine at least one of a patient condition or a type of the medical procedure; based on the at least one of the patient condition or the type of the medical procedure, automatically generating, by the processing circuitry, a report template; and presenting, by the processing circuitry, a user interface on a display, the user interface comprising the report template.

Example 20. The method of any of claims 15-19, further comprising: executing, by the processing circuitry, a computer vision model to determine an intraprocedural event; and based on determining the intraprocedural event, taking, by the processing circuitry, at least one action.

Example 21. The method of claim 20, wherein the at least one action comprises at least one of: informing, by the processing circuitry and via a user interface, a clinician to save a medical instrument; or generating, by the processing circuitry, a shipping label.

Example 22. The method of any of claims 15-21, further comprising: receiving, by the processing circuitry, a wake-up word via the second sensor; and based on receiving the wake-up word, beginning, by the processing circuitry, to record the audio data to memory.

Example 23. The method of claim 22, further comprising: determining, by the processing circuitry, that a predetermined time period from receiving the wake-up word has expired; and based on the determination that the predetermine time period from receiving the wake-up word has expired, stopping, by the processing circuitry, the recording the audio data to the memory.

Example 24. The method of any of claims 15-23, further comprising: executing, by the processing circuitry, a machine learning model to determine a patient condition; and presenting, by the processing circuitry, a user interface on a display, the user interface comprising the determined patient condition.

Example 25. The method of any of claims 15-24, further comprising sharing, by the processing circuitry, at least one of the video data or the audio data with another person or a group of people.

Example 26. The method of any of claims 15-25, further comprising: receiving, by the processing circuitry and via a user interface, performance information relating to the medical procedure; associating, by the processing circuitry, the performance information with one or more clinicians that performed the medical procedure; and storing, by the processing circuitry, the performance information in memory.

Example 27. The method of any of claims 15-26, further comprising: uploading, by the processing circuitry, at least one of the video data or the audio data to a server; editing, by the processing circuitry, the video data to generate an excerpt of the video data; and storing, by the processing circuitry, the excerpt of the video data in memory.

Example 28. The method of any of claims 15-27, further comprising: executing, by the processing circuitry, a machine learning model to determine a patient condition; based on the patient condition, generating, by the processing circuitry, a recommendation for a clinical trial for the patient; and presenting, via a user interface, the recommendation to a clinician.

Example 29. A non-transitory computer-readable storage medium storing instructions, which when executed cause processing circuitry to: receive video data from one or more first sensors, the video data being captured during a medical procedure of a patient; receive audio data from one or more second sensors, the audio data being captured during the medical procedure of the patient; and register the video data with the audio data.

Various examples have been described. These and other examples are within the scope of the following claims.

1. A medical system comprising:
memory configured to store video data and audio data, the video data and audio data being captured during a medical procedure of a patient; and
processing circuitry communicatively coupled to the memory, the processing circuitry being configured to:
receive the video data from one or more first sensors;
receive the audio data from one or more second sensors; and
register the video data with the audio data.

2. The medical system of claim 1, wherein as part of registering the video data with the audio data, the processing circuitry is further configured to:
apply one or more video timestamps to the video data; and
apply one or more audio timestamps to the audio data.

3. The medical system of claim 1, wherein the processing circuitry is further configured to present a user interface on a display, wherein the user interface comprises a plurality of drop-down menus, each of the plurality of drop-down menus having a limited number of selection options, the selection options being configured to categorize at least one of the medical procedure or a patient condition.

4. The medical system of claim 1, wherein the video data is first video data and wherein the processing circuitry is further configured to:
receive second video data from one or more third sensors;
register the second video data with the first video data; and
overlay the first video data and the second video data on a display, wherein one or more third sensors are of a different type than the one or more first sensors.

5. The medical system of claim 1, wherein the processing circuitry if further configured to:
execute a machine learning model to determine at least one of a patient condition or a type of the medical procedure;
based on the at least one of the patient condition or the type of the medical procedure, automatically generate a report template; and
present a user interface on a display, the user interface comprising the report template.

6. The medical system of claim 1, wherein the processing circuitry is further configured to:
execute a computer vision model to determine an intraprocedural event; and
based on determining the intraprocedural event, take at least one action.

7. The medical system of claim 6, wherein the at least one action comprises at least one of:
informing, via a user interface, a clinician to save a medical instrument; or
generating a shipping label.

8. The medical system of claim 1, wherein the processing circuitry is further configured to:
receive a wake-up word via the one or more second sensors; and
based on receiving the wake-up word, begin recording the audio data to the memory.

9. The medical system of claim 8, wherein the processing circuitry is further configured to:
determine that a predetermined time period from receiving the wake-up word has expired; and
based on the determination that the predetermine time period from receiving the wake-up word has expired, stop recording the audio data to the memory.

10. The medical system of claim 1, wherein the processing circuitry is further configured to:
execute a machine learning model to determine a patient condition; and
present a user interface on a display, the user interface comprising the determined patient condition.

11. The medical system of claim 1, wherein the processing circuitry is further configured to share at least one of the video data or the audio data with another person or a group of people.

12. The medical system of claim 1, wherein the processing circuitry is further configured to:
receive, via a user interface, performance information relating to the medical procedure;
associate the performance information with one or more clinicians that performed the medical procedure; and
store the performance information in the memory.

13. The medical system of claim 1, wherein the processing circuitry is further configured to:
upload at least one of the video data or the audio data to a server;
edit the video data to generate an excerpt of the video data; and
store the excerpt of the video data in the memory.

14. The medical system of claim 1, wherein the processing circuitry if further configured to:
execute a machine learning model to determine a patient condition;
based on the patient condition, generate a recommendation for a clinical trial for the patient; and
present the recommendation, via a user interface, to a clinician.

15. A method comprising:
receiving, by processing circuitry, video data from one or more first sensors, the video data being captured during a medical procedure of a patient;
receiving, by processing circuitry, audio data from one or more second sensors, the audio data being captured during the medical procedure of the patient; and
registering, by the processing circuitry, the video data with the audio data.

16. Non-transitory computer-readable storage media storing instructions, which when executed cause processing circuitry to:
receive video data from one or more first sensors, the video data being captured during a medical procedure of a patient;
receive audio data from one or more second sensors, the audio data being captured during the medical procedure of the patient; and
register the video data with the audio data.

17. The method of claim 15, wherein registering the video data with the audio data comprises: applying, by the processing circuitry, one or more video timestamps to the video data; and applying, by the processing circuitry, one or more audio timestamps to the audio data.

18. The method of claim 15, further comprising presenting, by the processing circuitry, a user interface on a display, wherein the user interface comprises a plurality of drop-down menus, each of the plurality of drop-down menus having a limited number of selection options, the selection options being configured to categorize at least one of the medical procedure or a patient condition.

19. The method of claim 15, wherein the video data is first video data and wherein the method further comprises: receiving, by the processing circuitry, second video data from one or more third sensors; registering, by the processing circuitry, the second video data with the first video data; and overlaying, by the processing circuitry, the first video data and the second video data on a display, wherein the one or more third sensors are of a different type than the one or more first sensors.

20. The method of claim 15, further comprising: executing, by the processing circuitry, a machine learning model to determine at least one of a patient condition or a type of the medical procedure; based on the at least one of the patient condition or the type of the medical procedure, automatically generating, by the processing circuitry, a report template; and presenting, by the processing circuitry, a user interface on a display, the user interface comprising the report template.
